(19) United States
(12) Patent Application Publication
(10) Pub. No.: US 2023/0288305 A1
(43) Pub. Date: Sep. 14, 2023

(54) ON-THE-FLY POLYMER QUALITY TESTING DEVICE

(71) Applicant: SAUDI ARABIAN OIL COMPANY, Dhahran (SA)

(72) Inventors: Abdulaziz AlKhateeb, Al-Khobar (SA); Waleed Abdulaziz Dokhon, Ulaya Khobar (SA); Ziyad Kaidar, Qatif, Alsadafah (SA); Ming Han, Dhahran (SA); Abdulkareem Alsofi, Dhahran (SA)

(73) Assignee: SAUDI ARABIAN OIL COMPANY, Dhahran (SA)

(21) Appl. No.: 17/585,366

(22) Filed: Jan. 26, 2022

(51) Int. Cl. G01N 11/04; G01N 1/20

(52) U.S. Cl. CPC G01N 11/04; G01N 1/20; G01N 33/28

(57) ABSTRACT

An assembly includes a bypass line having a first connection end and a second connection end, a device base positioned along the bypass line, an upstream valve positioned along the bypass line between the device base and the first connection end, and a downstream valve positioned along the bypass line between the device base and the second connection end. The device base includes a first opening to the bypass line, a second opening to the bypass line, and a support structure supporting at least one of the first and second openings.

### BACKGROUND

Enhanced oil recovery (EOR) methods may be used to extract oil from a reservoir that may not otherwise be extractable using conventional primary methods of recovery. EOR operations may generally include injecting a fluid (e.g., a gas, a polymer mixture, or a liquid) down an injection well to alter the downhole formation pressure and/or improve oil displacement or fluid flow in the reservoir. Examples of types of EOR operations include chemical flooding (e.g., alkaline or micellar polymer flooding), miscible displacement (e.g., injection of a miscible gas such as CO₂, hydrocarbon gases, or mixtures thereof), and thermal recovery (e.g., a steamflood or in-situ combustion).

EOR injectants may be designed to provide selected wetting characteristics in the formation in order to improve recovery of hydrocarbons from the formation. For example, a polymer solution may be designed (e.g., including polymer amount, polymer chain lengths, and polymer type) for a chemical flooding operation to achieve a desired performance downhole, such as to effect downhole pressure, pore channel plugging, wettability, viscosity, and interfacial properties. Changes in the polymer solution composition (e.g., from polymer degradation or harsh downhole environments) may affect the overall EOR operation performance.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to testing assemblies that include a bypass line having a first connection end and a second connection end, a device base positioned along the bypass line, an upstream valve positioned along the bypass line between the device base and the first connection end, and a downstream valve positioned along the bypass line between the device base and the second connection end. The device base may include a first opening to the bypass line, a second opening to the bypass line, and a support structure supporting at least one of the first and second openings (e.g., in a relative axial position to one another).

In another aspect, embodiments disclosed herein relate to systems that include a flow line fluidly connecting an injection pump to a wellhead, a production line fluidly connected to the wellhead, a flow divider valve positioned along the flow line between the injection pump and the wellhead, a bypass line extending from the flow divider valve to a downstream connection to the production line, an upstream valve and a downstream valve positioned along the bypass line, a device base positioned along the bypass line between the upstream valve and the downstream valve, and a removable apparatus installed in the device base fluidly connecting an upstream portion of the bypass line to a downstream portion of the bypass line.

In yet another aspect, embodiments disclosed herein relate to methods that include connecting a testing assembly to a flow line connecting an injection pump to a wellhead and to a production line. The testing assembly may include a bypass line extending from a flow divider valve along the flow line to a downstream connection along the production line, an upstream valve and a downstream valve positioned along the bypass line, a device base positioned along the bypass line between the upstream valve and the downstream valve, and a removable apparatus installed in the device base. Methods may further include flowing a fluid through the flow line and the bypass line, closing the upstream valve and the downstream valve to seal the fluid in a portion of the bypass line, and testing a property of the fluid sealed in the portion of the bypass line.

Other aspects and advantages will be apparent from the following description and the appended claims.

### BRIEF DESCRIPTION OF DRAWINGS

Wherever possible, like or identical reference numerals are used in the figures to identify common or the same elements. The figures are not necessarily to scale and certain features and certain views of the figures may be shown exaggerated in scale for purposes of clarification.

FIG. 1 shows a testing system according to embodiments of the present disclosure.

FIG. 2 shows an example of a device base according to embodiments of the present disclosure.

FIGS. 3 and 4 show an example of a removable apparatus assembled to a device base according to embodiments of the present disclosure.

FIG. 5 shows another example of a removable apparatus assembled to a device base according to embodiments of the present disclosure.

FIG. 6 shows another example of a removable apparatus assembled to a device base according to embodiments of the present disclosure.

FIG. 7 shows a method according to embodiments of the present disclosure.

### DETAILED DESCRIPTION

Embodiments of the present disclosure are described below in detail with reference to the accompanying figures. In the following detailed description, numerous specific details are set forth in order to provide a more thorough understanding of the claimed subject matter. However, it will be apparent to one having ordinary skill in the art that the embodiments described may be practiced without these specific details. In other instances, well-known features have not been described in detail to avoid unnecessarily complicating the description.

Embodiments described herein generally relate to on-the-fly polymer quality testing, which may be used to ensure the quality of polymer solutions passing through components in an injection facility at the surface of a well, e.g., prior to injection into a wellhead. For example, a testing assembly may be assembled along a flow line fluidly connecting an injection pump to the wellhead, where a polymer solution may be directed from the flow line and into the testing assembly to test the polymer quality in the fluid.

Polymer macromolecules in solution may be subject to mechanical degradation when flowing through various types of apparatuses (e.g., an injection pump, valves, chokes, pipeline connections, and/or a static mixer) during injection from surface facilities to a well, such as in a polymer flooding operation or other injection operation. Testing assemblies and methods disclosed herein provide a way to test the quality of a polymer solution after flowing through such apparatuses. Thus, such testing may be performed to determine if the tested apparatuses sufficiently avoid degrading the polymers and to assure the success of the injection operation (e.g., polymer flooding operation).

Testing assemblies disclosed herein may include a long bypass line that has a device base integrated along the bypass line. The device base may act as a universal component receptacle, where various types of apparatuses, such as valves, chokes, and static mixers, may be removably installed in the device base. When installed in a device base, an apparatus may provide a continuous flow path through the bypass line. In other words, an apparatus may be removably installed in a device base, such that when installed, a fluid may flow along a continuous flow path formed through both the bypass line and the apparatus, from an upstream portion of the bypass line, through the apparatus, and to a downstream portion of the bypass line.

When a polymer solution flows through an apparatus installed in the device base, the apparatus may agitate, grind, extrude, or exert other shearing forces on polymers in the solution, which may break the polymer molecule and cause mechanical degradation of the polymer. Such effects may be determined by testing the quality of the polymer solution after flowing through the apparatus. In other words, an apparatus may be removably installed in a device base of a testing assembly, and polymer quality of a fluid flowing through the installed apparatus may be tested in order to determine the effects on a polymer flowing through the apparatus. The polymer quality of the fluid may be tested from a testing site provided along the device base and/or the bypass line in a testing assembly, where a sample of the fluid may be taken to have one or more of its properties measured. While various testing procedures may be used to test the polymer quality in a fluid from a testing site, viscosity testing is described herein as an example of a way to evaluate polymer quality. For example, viscosity may be dependent on a molecular weight distribution of a polymer in solution. Correlations made between a polymer solution viscosity and the polymer’s molecular weight or chain length may be used to determine an amount of polymer degradation occurring in the polymer solution from an initial viscosity of the polymer solution.

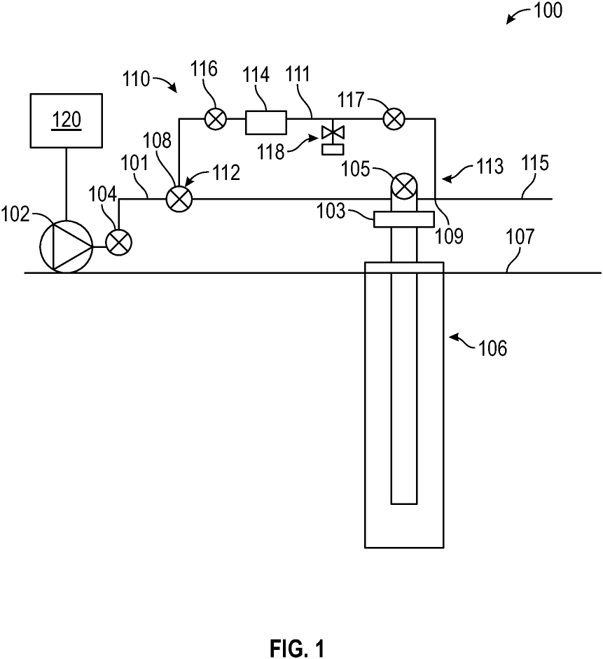

FIG. 1 shows an example of a system 100 using a testing assembly 110 according to embodiments of the present disclosure. The system may include a flow line 101 fluidly connecting an injection pump 102 to a wellhead 103 on a well 106, where a fluid may be pumped from an injection tank 120 (or other fluid source) to the well via the injection pump 102. The flow line 101 may be a pipe (e.g., a steel pipe) having various valves and/or other flow control devices provided along the length of the flow line 101, which may be used to control the flow of a fluid through the flow line 101. For example, a flow line valve 104 may be provided along the flow line 101, which may allow or prevent fluid flow between the wellhead 103 and the injection pump 102. Additionally, the flow line 101 may be connected to the wellhead 103 via a wellhead valve 105, which may be opened to allow fluid flow (or closed to prevent fluid flow) into or out of the wellhead 103. The wellhead valve 105 may be provided as part of the wellhead 103 (e.g., on a Christmas tree). In the embodiment shown, the flow line 101 may be used to inject fluid into the well 106, for example, in a chemical flooding operation (e.g., polymer flooding, surfactant-polymer flooding, alkaline-surfactant-polymer flooding) of the well 106.

Wellheads 103 may have multiple valves and multiple flow lines connected to the wellhead 103 to control the flow of fluids into and out of the well 106. For example, in the embodiment shown in FIG. 1, a production line 115 may also be connected to the wellhead 103, where the production line 115 may be used for directing fluids produced from or exiting the well 106 to a location at the surface 107 of the well 106. In some embodiments, the production line 115 may be a disposal line, where fluid that was injected into the well 106 may be directed through the production line 115 after returning to the surface and being ejected from the well 106. One or more different wellhead valves may be used to control the flow of fluid from the well to the production line 115, where such valves may be referred to as production line valves.

A testing assembly 110 according to embodiments of the present disclosure may be connected to the flow line 101 between the flow line valve 104 and the wellhead valve 105. Thus, closure of the flow line valve 104 may prevent fluid flow from a fluid source (e.g., tank 120) to the testing assembly 110. The testing assembly 110 may be connected at a first connection end 112 to the flow line 101 via a flow divider valve 108. The flow divider valve 108 may be positioned along the flow line 101 between the injection pump 102 and the wellhead 103, and may be used to divert the flow of fluid from the flow line 101 to the testing assembly 110. For example, the flow divider valve 108 may be a ball valve or other three-way valve, which in a first position, may close fluid flow to the flow line 101 and open fluid flow to the testing assembly, and in a second position, may open fluid flow to the flow line 101 and close fluid flow to the testing assembly.

According to embodiments of the present disclosure, a testing assembly 110 may include a bypass line 111 having a first connection end 112 and a second connection end 113 at opposite axial ends of the pipe. Pipe used to form the bypass line 111 may be made of steel and/or other material designed to resist corrosion from an anticipated fluid flowing therethrough. In some embodiments, the bypass line 111 may have an inner diameter ranging from a lower limit selected from 0.5 inch, 1 inch, or 2 inches to an upper limit selected from 2 inches, 5 inches, or more, where any lower limit may be used in combination with any upper limit. For example, a pipe having a common inner diameter of 1.5 inches may be used to form the bypass line 111. In some embodiments, the bypass line 111 may have a length (measured from the first connection end 112 to the second connection end 113) that is greater than 15 meters, greater than 50 meters, or greater than 70 meters, depending, for example, on the system 100 the testing assembly 110 is assembled to.

The bypass line 111 may extend from the flow divider valve 108 to a connection 109 to the production line 115, where the connection 109 may be located downstream from the wellhead 103. The connection 109 may be at a valved junction (e.g., via a ball valve or other three-way valve) or at a pipe junction without a valve.

A device base 114 may be positioned along the bypass line 111, for example, along a central portion of the bypass line 111. An upstream valve 116 may be positioned along the bypass line 111 between the device base 114 and the first connection end 112, and a downstream valve 117 may be positioned along the bypass line 111 between the device base 114 and the second connection end 113 of the bypass line. In such manner, the device base 114 may be positioned along the bypass line 111 between the upstream valve 116 and a downstream valve 117.

Additionally, the testing assembly 110 may have a testing site 118 provided along the bypass line 111 or with the device base 114. In the embodiment shown in FIG. 1, the testing site 118 may be provided along the bypass line 111, between the device base 114 and the downstream valve 117. The testing site 118 may include a sampling outlet provided in the bypass line 111, which may be selectively opened and closed to release an amount of fluid from the bypass line 111 for testing. In some embodiments, the testing site 118 may include a sampling line fluidly connected to the bypass line 111, where fluid may be flowed from the bypass line 111 through the sampling line to be tested. A sampling valve, such as a pressure relief valve, may be provided at a sampling outlet or along a sampling line to selectively allow fluid to drain from the bypass line 111 and be tested.

By providing a testing site 118 along a testing assembly 110 fluidly connected to and branched off a flow line 101, fluid from the flow line 101 may be rerouted to the testing assembly 110 and tested at the testing site 118 offline from operations using the flow line 101 (e.g., by closing one or more valves along the flow line 101 and/or bypass line 111). Additionally, by using methods disclosed herein for testing fluid from a testing assembly, unwanted mechanical degradation of polymers in the fluid may be minimized while taking samples of the fluid for testing. For example, in some embodiments, fluid may be sealed in the bypass line 111 (e.g., by closing valves around and/or along the bypass line), a sample of the fluid may be drained from a sampling outlet provided along the bypass line 111 at a location downstream from the device base 114, and the viscosity of the sample may be measured (e.g., using a viscometer). Sample testing may be done on site at the testing site 118, or a fluid sample collected from the testing site 118 may be brought to a laboratory or other off-site testing equipment for fluid testing.

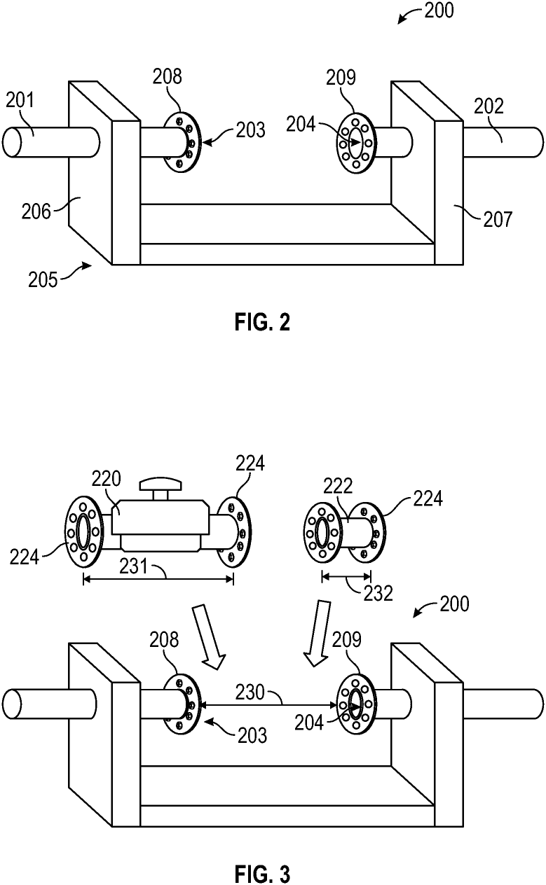

Referring now to FIG. 2, FIG. 2 shows an example of a device base 200 that may be used in a testing assembly according to embodiments of the present disclosure. The device base 200 may be connected to and form a part of a bypass line (e.g., 111 in FIG. 1) via a first bypass connection 201 and a second bypass connection 202. The first bypass connection 201 may be connected to an upstream portion of the bypass line and the second bypass connection 202 may be connected to a downstream portion of the bypass line, for example, using pipe connections (e.g., clamps or flanged connections). The device base 200 may further include a first opening 203 to the bypass line, where the first opening 203 is fluidly connected to the first bypass connection 201, and a second opening 204 to the bypass line, where the second opening 204 is fluidly connected to the second bypass connection 202. For example, in the embodiment shown in FIG. 2, the first and second bypass connections 201, 202 may be pipe segments, where one axial end of each pipe segment may be connected to the bypass line and the opposite axial end provides the opening (first and second openings 203, 204) to the bypass line.

The device base 200 may further include a support structure 205, which may be used to support at least one of the first and second openings 203, 204. For example, in some embodiments, a support structure may be a single piece component having two or more arms, brackets, or other support element that may support or hold the first and second openings 203, 204 in a position relative to each other. In some embodiments, such as shown in FIG. 2, a support structure 205 may include two or more support elements 206 assembled to a base 207, where the support elements 206 may hold the first and second openings 203, 204 in a position relative to each other. Various types and geometries for the support structure 205 may be envisioned. In some embodiments, the support structure 205 may hold the first and second openings 203, 204 in a position relative to each other where the first and second openings 203, 204 are in axial alignment, such as shown in FIG. 2. In some embodiments, a support structure may hold the first and second openings 203, 204 in a position where they are axially offset.

According to embodiments of the present disclosure, a removable apparatus may be installed in the device base 200 to fluidly connect the first opening 203 to the second opening 204. Connection elements 208, 209 may be connected around the first and second openings 203, 204, or connection elements may be integrally formed around the first and second openings 203, 204, to connect the first and second openings 203, 204 to a removable apparatus. For example, as shown in FIG. 2, flange connections 208, 209 may be provided around the first and second openings 203, 204.

Removable apparatuses may include various types of components to be tested for determining their effects on degradation of polymers in a fluid flowing through the component. For example, removable apparatuses may include valves, e.g., a gate valve, ball valve, choke valve, globe valve, check valve, plug valve, butterfly valve, or torque valve, a static mixer, or a choke. Removable apparatuses may also include high pressure apparatuses, for example, apparatuses capable of holding a pressure up to about 2,000 psi.

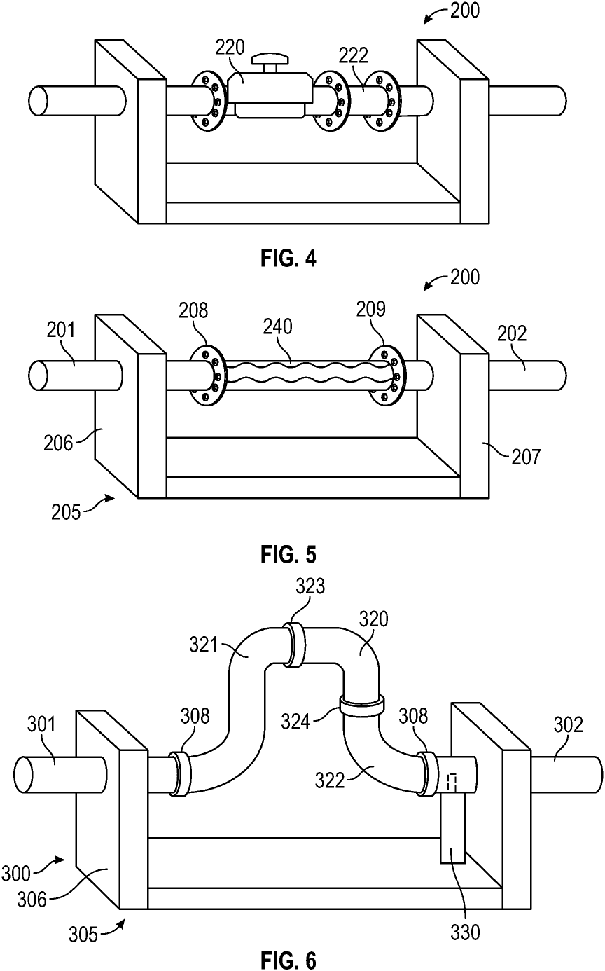

For example, FIGS. 3 and 4 show an example of removable apparatuses that may be installed in the device base 200 shown in FIG. 2. The removable apparatuses may include a valve 220 and an extension piece 222, where the extension piece 222 may be used to connect the valve 220 to an opening (e.g., 202 or 203) in the device base 200. For example, when a device base 200 has a gap 230 formed between the first and second openings 203, 204 to the bypass line that is greater than a length 231 of the valve 220, one or more extension pieces 222 may be used to bridge the remaining portion of the gap 230 for fluidly connecting the valve 220 to the first and second openings 203, 204. In the embodiment shown, one extension piece 222 may be used to connect the valve 220 to an opening of the device base 200, where the extension piece 222 may have a length 232 that is equal to the difference between the length of the gap 230 and the length 231 of the valve 220. The extension piece 222 may be a pipe having a flow path formed through its length 232 that may fluidly connect a flow path through the valve 220 to the first or second opening 203, 204 to the bypass line.

As shown in FIG. 4, the valve 220 and the extension piece 222 may be connected together and to the device base 200 using flange connections 208, 209, and 224. However, other types of pipe connections may be used, such as clamps. In some embodiments, sealing elements may be used with pipe connections to seal the connection between the first and second openings 203, 204 to the removable apparatus(es), e.g., an o-ring may be provided between two flanges in a flanged connection. When the removable apparatuses (valve 220 and extension piece 222) are installed in the device base 200, a continuous flow path may be formed along the bypass line, device base 200, and removable apparatuses 220, 222, such that fluid may flow from a bypass line to the device base 200, through the first opening 203, through the valve 220, through the extension piece 222, and through the second opening 204, and return to the bypass line.

The valve 220 may be selected from valves of interest for use in a system where a polymer solution would flow therethrough (e.g., in flooding operation for a well system). By installing the valve 220 in the device base 200 and flowing a polymer solution therethrough, the effects of the valve 220 on the polymer solution may be determined by testing a sample of the polymer solution after flowing through the valve 220. The valve 220 may be selected from various types of valves known in the art and may be replaced by other valves for comparison of the effects on the polymer solution. In some embodiments, the valve 220 may be replaced by a different type of apparatus, such as a choke or a static mixer 240.

For example, FIG. 5 shows an example of a different type of removable apparatus installed in the device base 200 shown in FIG. 2, where the removable apparatus is a static mixer 240. A static mixer 240 is an apparatus that may be used for the continuous mixing of polymer solutions (e.g., a surfactant polymer mixture). The static mixer 240 may include a cylindrical tube containing flow disrupters (e.g., plates in a plate-type static mixer or a helical baffle in a helical-type static mixer), where the flow disrupters may extend into the flow path formed through the tube to disrupt the flow of and mix fluids flowing through the static mixer. The flow path formed through the static mixer 240 may have an inner diameter that is the same as or different than the inner diameter of the bypass line, and may range, for example, from 0.5 inches to 2 inches (e.g., 1 inch inner diameter or 1.5 inch inner diameter). Additionally, in some embodiments, a static mixer 240 may have a length greater than 1 foot (e.g., ranging from 3 to 4 feet long). In some embodiments, a static mixer 240 may be made of glass-lined steel.

When the static mixer 240 is installed in the device base 200, a polymer solution may be flowed through the bypass line and the connected static mixer 240, where the static mixer 240 may mix the polymer solution as it flows therethrough. The polymer solution may then be tested downstream from the static mixer 240 at a testing site to test the quality of the polymer mixture (e.g., measuring the viscosity of the polymer mixture as an indicator of polymer degradation). In such manner, the effect of the static mixer 240 on a polymer solution may be determined. By using the device base 200 according to embodiments of the present disclosure to test the static mixer 240, other types of removable apparatuses may be easily installed and replaced in the device base 200 to test the effects on a polymer solution from each removable apparatus.

FIG. 6 shows an additional example of a removable apparatus installed in device base according to embodiments of the present disclosure. However, other combinations and configurations may be envisioned according to embodiments of the present disclosure.

In FIG. 6, a device base 300 may include a support structure 305 having multiple support elements 306 holding bypass connections 301, 302 in an axially aligned position. However, other configurations of support elements may be envisioned to hold bypass connections 301, 302 in a selected axial position relative to each other. A removable apparatus, e.g., a choke 320, may be installed in the device base 300 and fluidly connected to the bypass connections 301, 302 via connections 308, such that fluid may be flowed through a bypass line to the device base 300 and through the installed removable apparatus 320. In the embodiment shown, the choke 320 may have a turn, e.g., a 90-degree turn.

When a removable apparatus having a turn or other non-linear shape is to be installed in a device base 300 having axially aligned bypass connections 301, 302, such as shown in FIG. 6, one or more extension pieces 321, 322 may be used to connect the removable apparatus to the bypass connections 301, 302. For example, in the embodiment shown in FIG. 6, a first extension piece 321 may have multiple turns and connect a first bypass connection 301 to a first end of the choke 320 via a first connection 323, and a second extension piece 322 may have one turn and connect a second bypass connection 302 to a second end of the choke 320 via a second connection 324. Once the choke 320 is fluidly connected to the device base 300 via the extension pieces 321, 322, a fluid may be flowed from an upstream portion of the bypass line, through the first bypass connection 301, through the first extension piece 321, through the choke 320, through the second extension piece 322, and through the second bypass connection 302 to a downstream portion of the bypass line.

According to embodiments of the present disclosure, a viscometer 330 may be provided at a testing site along the device base 300 to measure the viscosity of fluid flowing through the device base 300. For example, in the embodiment shown, a viscometer 330 may be provided with the second bypass connection 302 of the device base 300. The viscometer 330 may be an in-line type viscometer, which may be inserted partially into the flow path through the second bypass connection 302 to measure the viscosity of fluid flowing therethrough. In other embodiments, a different type of testing device may be provided with the device base 300 to measure the quality of a polymer solution.

By providing a testing site (e.g., viscometer 330) downstream of the removable apparatus (e.g., choke 320) being analyzed, the effect of the removable apparatus on the quality of the polymer solution flowing therethrough may be determined. However, in some embodiments, a viscometer (or other testing device) may be fluidly connected to an upstream portion of the device base (e.g., first bypass connection 301). In some embodiments, a viscometer (or other testing device) may be fluidly connected to both an upstream portion (e.g., first bypass connection 301) and a downstream portion (e.g., second bypass connection 302) of the device base 300, which may be used to provide polymer quality data for a polymer solution before and after flowing through the removable apparatus, and which may be compared to determine changes in the polymer solution resulting from the removable apparatus.

Testing assemblies according to embodiments of the present disclosure may be used in combination with fluid systems to provide polymer quality testing for different apparatuses of interest for use with the fluid system. For example, testing apparatuses disclosed herein may be used with a well system to test the effects on a polymer solution from different apparatuses of interest for use in the well system. Methods of testing the effects of an apparatus on a polymer solution using testing assemblies according to embodiments of the present disclosure may include flowing the polymer solution through an apparatus being tested in the testing assembly and measuring at least one property (e.g., viscosity) of the polymer solution after flowing through the apparatus.

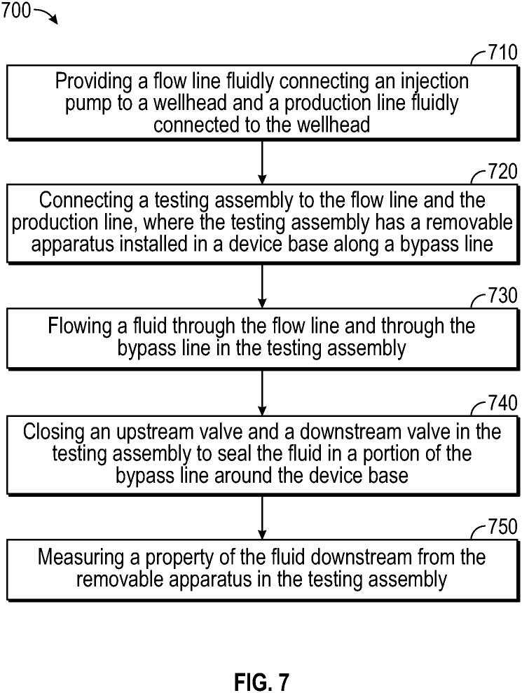

FIG. 7 shows an example of a method 700 for using a testing assembly according to embodiments of the present disclosure. One or more steps shown in FIG. 7 may be repeated or omitted. Additionally, steps shown in FIG. 7 may be described using elements shown in FIG. 1 as an example.

As shown, a method may include providing a flow line fluidly connecting one or more injection pumps to a wellhead and a production line or discharge line fluidly connected to a downstream flow path in the wellhead (step 710). A testing assembly according to embodiments of the present disclosure may be connected to the flow line at a first connection end of the testing assembly and to the production line at a second connection end of the testing assembly (step 720). In such manner, a testing assembly (e.g., 110 in FIG. 1) may be assembled to an operational well system. By assembling a testing assembly to an operational well system (e.g., to a flow line connecting an injection pump to a wellhead), operational conditions such as the injection rate, pumping pressure, temperature, etc. of the well system may be used and replicated when testing a polymer solution through the testing assembly.

Testing assemblies that may be used in methods according to embodiments of the present disclosure may include, for example, a bypass line (e.g., 111 in FIG. 1) extending from a flow divider valve (e.g., 108 in FIG. 1) on the flow line (e.g., 101) to a downstream connection along the production line, where the downstream connection is downstream from the wellhead (e.g., 103 in FIG. 1). An upstream valve (e.g., 116 in FIG. 1) and a downstream valve (e.g., 117 in FIG. 1) may be positioned along the bypass line, and a device base (e.g., 114 in FIG. 1) may be positioned along the bypass line between the upstream valve and the downstream valve. The device base may provide a first opening to the bypass line and a second opening to the bypass line, where a removable apparatus may be installed in the device base and fluidly connect the first opening to the second opening of the bypass line. In such manner, a continuous flow path through the bypass line may be formed when a removable apparatus is installed in the device base. As discussed herein, a removable apparatus may be an apparatus having a fluid flow path formed therethrough and may have a functionality to direct fluid therethrough in a certain way. For example, a removable apparatus may be a high pressure valve or choke.

When a testing assembly according to embodiments of the present disclosure is assembled to a flow line, a method may further include flowing a fluid through the flow line and the bypass line in the testing assembly (step 730) to the production line, where the tested fluid may be discharged or reused. For example, a polymer solution may be pumped through the flow line using one or more injection pumps and directed to a bypass line in a connected testing assembly via a flow divider valve. An amount of the fluid may be pumped through the bypass line sufficient to flush the bypass line with the fluid. For example, at least 2 barrels of the fluid (e.g., between 2 and 5 barrels) may be flowed through the bypass line to flush the bypass line. Fluid may be flushed through the bypass line and into a fluidly connected production line (e.g., 115 in FIG. 1). Flushing the bypass line with a fluid prior to testing the fluid may be done to prevent remnants of other material in the bypass line from effecting the testing.

After flowing a fluid through the flow line and the bypass line in the testing assembly, an upstream valve and a downstream valve in the testing assembly may be closed to seal a portion of the fluid in a portion of the bypass line around the device base (step 740). In some embodiments, prior to closing the upstream valve and the downstream valve, the pumping rate of the fluid may be reduced and a wellhead valve (e.g., 105 in FIG. 1) to the wellhead may be closed to stop fluid flow into the wellhead.

After stopping the flow of fluid through the bypass line, a property of the fluid sealed in the bypass line may be measured (step 750). For example, according to embodiments of the present disclosure, a sample of the fluid may be drained from a sampling outlet provided along the bypass line between the device base and the downstream valve (downstream of the device base and installed removable apparatus). Once the sample of fluid is drained, a property of the fluid sample may be measured, such as the viscosity of the fluid sample. Other testing devices may be provided at a testing site downstream from the installed removable apparatus to test a property of fluid that flowed through the removable apparatus. Further, other methods may be used to measure a property of the fluid within the testing assembly, including methods that drain a fluid sample prior to testing and methods that measure a fluid property within the bypass line (without draining a fluid sample from the bypass line).

The measured property of the fluid may be used to determine a quality of polymers in the fluid. For example, the viscosity of a polymer solution may be measured at a testing site downstream from the removable apparatus installed in the testing assembly, where the viscosity may indicate an amount of polymer degradation occurring from the polymer solution flowing through the removable apparatus.

According to embodiments of the present disclosure, a measured viscosity of a polymer solution downstream from an installed removable apparatus may be compared with an initial viscosity of the polymer solution in order to determine an amount of polymer degradation resulting from its flow between the point where the polymer solution has the initial viscosity to the point where the polymer solution has the downstream viscosity measurement. In some embodiments, an initial viscosity may be determined prior to pumping the polymer solution from an injection tank to a well system via injection pumps. In some embodiments, an initial viscosity may be measured at a location along the testing assembly upstream from the removable apparatus (e.g., in an upstream portion of a bypass line, upstream from an installed removable apparatus).

By comparing a downstream measured viscosity of the polymer solution to an initial viscosity of the polymer solution, the degradation level of the polymer solution may be identified resulting from the flow of the polymer solution between a location where the polymer solution has the initial viscosity and the downstream location where the measured viscosity is taken. For example, in some embodiments, an initial viscosity of a polymer solution may be determined for the polymer solution in an injection tank. The polymer solution may then be pumped via injection pump(s) to a testing assembly, where a downstream viscosity may be measured after the polymer solution has flowed through an installed removable apparatus. In such embodiments, an amount of polymer degradation resulting from the polymer solution flowing from the injection tank to the removable apparatus may be determined by comparing the initial viscosity to the downstream viscosity.

In some embodiments, an initial viscosity of a polymer solution may be measured upstream of an installed removable apparatus in an upstream portion of a testing assembly, and a downstream viscosity of the polymer solution may be measured downstream of the installed removable apparatus in a downstream portion of the testing assembly. In such embodiments, an amount of polymer degradation resulting from the polymer solution flowing through the removable apparatus may be determined by comparing the initial viscosity to the downstream viscosity.

Thus, by using methods according to embodiments of the present disclosure to determine an amount of polymer degradation resulting from flow through a system, the strength of the polymer solution in the field may be identified, including identifying how well the polymer solution can hold different shears using different removable apparatuses.

Further, according to embodiments of the present disclosure, methods disclosed herein may include replacing a first removable apparatus with a different, second removable apparatus in a device base provided in a testing assembly. By using testing assemblies according to embodiments of the present disclosure, where removable apparatuses may be interchangeably installed in the device base provided along the testing assembly, fluid testing methods according to embodiments of the present (e.g., as shown in FIG. 7) may easily be performed to test the effects from multiple different apparatuses.

Additional examples of methods according to embodiments of the present disclosure are provided below.

### Example 1

An example testing procedure according to embodiments of the present disclosure is provided below and described with respect to the system shown in FIG. 1. However, similar testing procedures may be performed on other systems having testing assemblies according to embodiments of the present disclosure.

A testing procedure for testing polymer quality of a polymer solution flowing through the well system 100 shown in FIG. 1 may include:

1) Open a flow line valve 104 (e.g., a production valve), which may allow the polymer solution to be pumped from a fluid source 120 via one or more injection pumps 102 through the flow line 101.

2) Open a flow divider valve 108 to divert flow from the flow line 101 to the bypass line 111 of a testing assembly 110.

3) Close a wellhead valve 105 to prevent fluid from an upstream portion of the flow line 101 from flowing to the wellhead 103.

4) Flush the bypass line 111 with the polymer solution (e.g., 2-5 barrels of the polymer solution), where the flushed polymer solution may be injected into the wellhead 103.

5) Reduce the pumping rate of the injection pump(s) 102 and close a production line 115 from the wellhead 103 (e.g., close a production line valve) at the same time.

6) Open wellhead valve 105 and close a downstream valve 117 in the testing assembly 110 at the same time in order to prevent the polymer solution from flowing from the bypass line 111 to the wellhead 103.

7) Close an upstream valve 116 in the testing assembly 110 in order to seal an amount of the polymer solution within the bypass line 111 between the upstream valve 116 and the downstream valve 117.

8) Release the pressure in the sealed portion of the bypass line 111 to 0 psi at a sampling point (testing site 118) and collect a sample of the polymer solution sample for viscosity measurement.

The sample viscosity measurement may be compared with an initial viscosity of the polymer solution (e.g., the viscosity of the polymer solution at the fluid source 120) to determine an amount of polymer degradation resulting from the flow of the polymer solution from the location of initial viscosity to the testing site 118.

Different removable apparatuses may be interchangeably installed in the device base 114 of the testing assembly 110 to test the effects of the different removable apparatuses on the amount of resulting polymer degradation in a polymer solution flowing therethrough.

### Example 2

A choke having a 1-inch inner diameter was installed in a device base of a testing assembly according to embodiments of the present disclosure. A surfactant-polymer mixed solution in seawater at concentration of 0.2% polymer and 0.7% surfactant respectively was passed through the device base and installed choke according to testing procedures described herein. Five barrels of the surfactant-polymer mixed solution were pumped through the testing assembly at a differential pressure of 200 psi. Sampling of the solution from the testing assembly was conducted at about 25° C. in 2 scenarios: 1) the choke was half opened and 2) the choke was fully opened. The initial viscosity of the surfactant-polymer solution was 72 cP before passing through the choke, which was measured using a Brookfield viscometer at a shear rate of 6 rpm. After flowing through the choke and sampled downstream of the choke, the downstream measured viscosity of the solution was 32 cP when the choke was half opened and 70 cP when the choke was fully opened. These results demonstrated that the viscosity of the polymer in solution was reduced by 44.4% through the half-opened choke, while the polymer degradation was very limited through the fully opened choke.

### Example 3

A torque valve having a 1-inch inner diameter was installed in a device base of a testing assembly according to embodiments of the present disclosure. A surfactant-polymer mixed solution in seawater at concentration of 0.2% polymer and 0.7% surfactant respectively was passed through the device base and installed torque valve according to testing procedures described herein. Five barrels of the surfactant-polymer mixed solution were pumped through the device at a differential pressure of 200 psi. Sampling of the solution from the testing assembly was conducted at about 25° C. in 2 scenarios: 1) the valve was half opened and 2) the valve was fully opened. The initial viscosity of the surfactant-polymer solution was 72 cP before passing through the valve, measured using a Brookfield viscometer at a shear rate of 6 rpm. A downstream viscosity of the solution measured at a testing site downstream from the valve was 10 cP when the valve was half opened and 70 cP when the valve was fully opened. These results demonstrated that the viscosity of the polymer in solution was reduced by 13.9% through the half-opened valve, while the polymer degradation was very limited through the fully opened valve.

While the present disclosure has been described with respect to a limited number of embodiments, those skilled in the art, having benefit of this disclosure, will appreciate that other embodiments may be devised which do not depart from the scope of the disclosure as described herein. Accordingly, the scope of the disclosure should be limited only by the attached claims.

What is claimed:

1. An assembly, comprising:
a bypass line having a first connection end and a second connection end;
a device base positioned along the bypass line, wherein the device base comprises:
a first opening to the bypass line;
a second opening to the bypass line; and
a support structure supporting at least one of the first and second openings;
an upstream valve positioned along the bypass line between the device base and the first connection end; and
a downstream valve positioned along the bypass line between the device base and the second connection end.

2. The assembly of claim 1, wherein the first connection end is connected to a flow divider valve positioned along a flow line to an injection pump.

3. The assembly of claim 1, wherein the second connection end is connected to a production line, and wherein the production line is fluidly connected to a wellhead.

4. The assembly of claim 1, further comprising a sampling outlet provided along the bypass line between the device base and the downstream valve.

5. The assembly of claim 1, further comprising a high pressure apparatus installed in the device base fluidly connecting the first opening to the second opening, wherein the high pressure apparatus is capable of holding a pressure up to about 2,000 psi.

6. The assembly of claim 5, wherein the high pressure apparatus is a valve.

7. The assembly of claim 5, wherein the high pressure apparatus is a static mixer.

8. The assembly of claim 1, wherein the device base further comprises a viscometer fluidly connected to at least one of the first opening and the second opening.

9. The assembly of claim 1, wherein the bypass line is greater than 50 meters long.

10. A system, comprising:
a flow line fluidly connecting an injection pump to a wellhead;
a production line fluidly connected to the wellhead;
a flow divider valve positioned along the flow line between the injection pump and the wellhead;
a bypass line extending from the flow divider valve to a downstream connection to the production line;
an upstream valve and a downstream valve positioned along the bypass line;
a device base positioned along the bypass line between the upstream valve and the downstream valve, wherein the device base comprises:
a first opening to the bypass line;
a second opening to the bypass line; and
a support structure supporting at least one of the first and second openings; and
a removable apparatus installed in the device base fluidly connecting the first opening to the second opening.

11. The system of claim 10, further comprising a wellhead valve on the wellhead, wherein the flow line is connected to the wellhead via the wellhead valve.

12. The system of claim 10, further comprising a production line valve on the wellhead, wherein the production line is fluidly connected to the wellhead via the production line valve.

13. The system of claim 10, further comprising a sampling line fluidly connected to the bypass line between the device base and the downstream valve.

14. The system of claim 10, wherein the removable apparatus is connected to the first opening and the second opening via flange connections.

15. The system of claim 10, wherein the removable apparatus comprises a choke having a turn.

16. A method, comprising:
providing a flow line fluidly connecting an injection pump to a wellhead;
providing a production line fluidly connected to the wellhead;
connecting a testing assembly to the flow line and the production line, wherein the testing assembly comprises:
a bypass line extending from a flow divider valve along the flow line to a downstream connection along the production line;
an upstream valve and a downstream valve positioned along the bypass line;
a device base positioned along the bypass line between the upstream valve and the downstream valve, wherein the device base comprises:
a first opening to the bypass line;
a second opening to the bypass line; and
a support structure supporting at least one of the first and second openings; and
a removable apparatus installed in the device base fluidly connecting the first opening to the second opening;
flowing a fluid through the flow line and the bypass line;
closing the upstream valve and the downstream valve to seal the fluid in a portion of the bypass line; and
testing a property of the fluid sealed in the portion of the bypass line.

17. The method of claim 16, further comprising, prior to closing the upstream valve and the downstream valve, reducing a pumping rate of the fluid and closing a wellhead valve to stop fluid flow from the wellhead to the production line.

18. The method of claim 16, wherein the testing comprises:
draining a sample of the fluid from a sampling outlet provided along the bypass line between the device base and the downstream valve; and
measuring a viscosity of the sample.

19. The method of claim 16, further comprising removing the removable apparatus and installing a second removable apparatus in the device base to fluid connect the first opening to the second opening.

20. The method of claim 16, wherein at least 2 barrels of the fluid is flowed through the bypass line prior to closing the upstream valve and the downstream valve.
